(19) United States
(12) Patent Application Publication
(10) Pub. No.: US 2024/0091425 A1
(43) Pub. Date: Mar. 21, 2024

(54) SYSTEMS AND METHODS FOR CONTROLLING BLOOD TEMPERATURE IN EXTRACORPOREAL BLOOD CIRCUITS

(71) Applicant: TC1 LLC, St. Paul, MN (US)

(72) Inventors: William Mock, Frisco, TX (US); Lukas Gratwohl, Zurich (CH)

(21) Appl. No.: 18/471,620

(22) Filed: Sep. 21, 2023

Related U.S. Application Data
(60) Provisional application No. 63/408,577, filed on Sep. 21, 2022.

(51) Int. Cl. A61M 1/36

(52) U.S. Cl. CPC A61M 1/3623

(57) ABSTRACT

An extracorporeal blood circuit is provided. The extracorporeal blood circuit includes a blood pump including an inlet, an outlet, and a rotor positioned between the inlet and the outlet, and a controller coupled to the blood pump. The controller is configured to drive rotation of the rotor using a voltage waveform and a current waveform, and adjust a phase angle between the voltage waveform and the current waveform to generate reactive power. The generated reactive power facilitates heating blood as the blood passes through the blood pump.

### CROSS REFERENCE TO RELATED APPLICATIONS

This application claims priority to provisional application serial No. 63/408,577, filed Sep. 21, 2022, which is incorporated herein by reference in its entirety.

### BACKGROUND OF THE DISCLOSURE

### a. Field of the Disclosure

The present disclosure relates generally to mechanical circulatory support systems, and more specifically relates to controlling blood temperature in extracorporeal blood circuits.

### b. Background

Many types of cardiac, cardiopulmonary, and pulmonary assist devices have been developed for applications in which a patient's heart and/or lungs are incapable of providing adequate circulation and/or gas exchange. For example, a patient suffering from acute heart failure may use an extracorporeal pump or circulatory support system that pumps blood out of and back into a patient's body. As another example, a patient suffering from cardiogenic shock after an invasive cardiothoracic procedure may use an extracorporeal pump or circulatory support system that provides perfusion assistance, blood oxygenation, and blood carbon dioxide removal. Extracorporeal circulatory support systems may also be used perioperatively, for example, to bypass the patient's circulatory system and provide perfusion of oxygenated blood while open-chest or other high-risk cardiothoracic surgery is performed.

In at least some known extracorporeal circulatory support systems, blood passing through the system cools at least a few degrees, due to the blood being outside of the patient's body. To counteract this cooling, at least some known systems attempt to warm or thermally isolate the patient, which requires additional equipment and resources. Further, at least some known systems attempt to thermally isolate the blood flow and/or actively warm tubing through which the blood flows. However, these systems also require additional equipment and resources.

Accordingly, a need exists for counteracting the cooling of blood in extracorporeal circulatory support systems, without requiring additional equipment and resources.

### SUMMARY OF THE DISCLOSURE

In one aspect, an extracorporeal blood circuit is provided. The extracorporeal blood circuit includes a blood pump including an inlet, an outlet, and a rotor positioned between the inlet and the outlet, and a controller coupled to the blood pump, the controller configured to drive rotation of the rotor using a voltage waveform and a current waveform, and adjust a phase angle between the voltage waveform and the current waveform to generate reactive power, wherein the generated reactive power facilitates heating blood as the blood passes through the blood pump.

In another aspect, a method of operating an extracorporeal blood circuit is provided. The method includes receiving, at an inlet of a blood pump, blood from a patient, driving, using a controller, rotation of a rotor of the blood pump to pump the blood from the inlet of the blood pump to an outlet of the blood pump, wherein the controller drives rotation of the rotor using a voltage waveform and a current waveform, and adjusting, using the controller, a phase angle between the voltage waveform and the current waveform to generate reactive power, wherein the generated reactive power facilitates heating the blood as the blood passes through the blood pump.

In yet another aspect, a controller for use in an extracorporeal blood circuit is provided. The controller includes a memory device, and a processing device coupled to the memory device, the processing device configured to drive rotation of a blood pump based on a voltage waveform and a current waveform, and adjust a phase angle between the voltage waveform and the current waveform to generate reactive power, wherein the generated reactive power facilitates heating blood as the blood passes through the blood pump.

### BRIEF DESCRIPTION OF THE DRAWINGS

FIG. 1 is an illustration of an extracorporeal circulatory support system connected to a patient's body.

FIG. 2 is a perspective view of one embodiment of an extracorporeal blood pump assembly suitable for use in the mechanical circulatory support system of FIG. 1.

FIG. 3 is a cross-sectional view of the extracorporeal blood pump assembly shown in FIG. 2 taken along line 3-3.

FIG. 4 is a diagram showing the relationship between active power, reactive power, and complex power.

FIG. 5 is a diagram illustrating the relationship between voltage, current, and active and reactive power.

### DETAILED DESCRIPTION OF THE DISCLOSURE

The present disclosure is directed to systems and methods including an extracorporeal blood circuit that includes a blood pump including an inlet, an outlet, and a rotor positioned between the inlet and the outlet, and a controller coupled to the blood pump. The controller is configured to drive rotation of the rotor using a voltage waveform and a current waveform, and adjust a phase angle between the voltage waveform and the current waveform to generate reactive power. The generated reactive power facilitates heating blood as the blood passes through the blood pump.

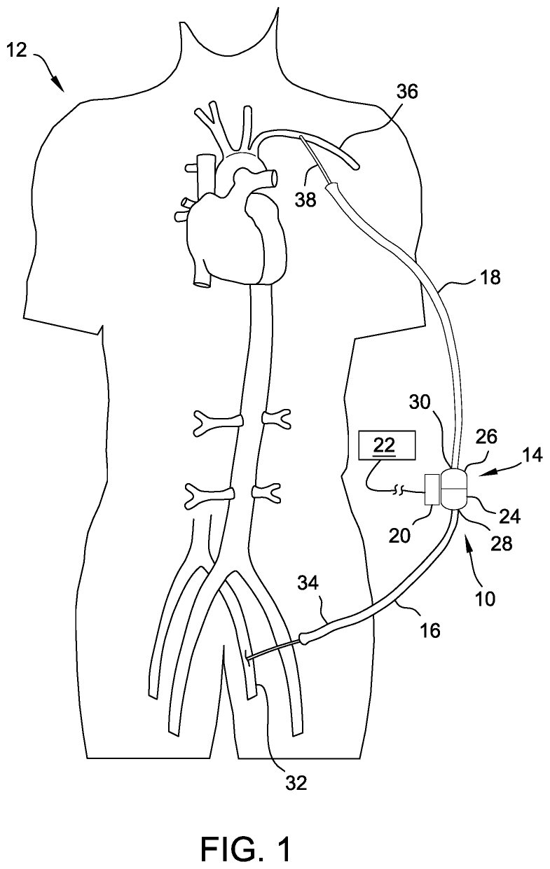

Referring now to the drawings, FIG. 1 is an illustration of an extracorporeal mechanical circulatory support system 10 connected a patient's 12 vasculature. The extracorporeal mechanical circulatory support system 10 includes an extracorporeal blood pump assembly 14, an inflow or first conduit 16, an outflow or second conduit 18, a controller 20, and a power source 22.

The blood pump assembly 14 includes a blood pump 24, an oxygenator (e.g., for use in extracorporeal membrane oxygenation (ECMO)) 26, and an inlet 28 and an outlet 30 for connection of flexible conduits thereto. The blood pump assembly 14 may include any suitable type of pump that enables the blood pump assembly 14 to function as described herein, including, for example and without limitation, an axial rotary pump and a centrifugal rotary pump. The oxygenator 26 may include an oxygenator membrane (not shown) configured to increase the oxygen concentration and/or decrease the carbon dioxide concentration of blood pumped through the blood pump assembly 14. Further, the system 10 may also include a purge valve (not shown) to release air or other gases present within the system 10. The purge valve can be connected, for example, to the outflow conduit 18, or may be integrated within the oxygenator 26 (e.g., at an outlet of the oxygenator 26). Although the embodiments disclosed herein are described in terms of an oxygenator system, those of skill in the art will appreciate that the systems and methods disclosed herein may be used in other systems (e.g., ventricular assist device systems that do not include an oxygenator).

The blood pump assembly 14 is connected to the patient's vasculature through the inflow conduit 16 and the outflow conduit 18. More specifically, the inlet 28 of the blood pump assembly 14 is connected to the inflow conduit 16, and the outlet 30 of the blood pump assembly 14 is connected to the outflow conduit 18. The inflow conduit 16 is connected to the patient's vasculature, specifically, to a first peripheral blood vessel 32 in the illustrated embodiment, by way of a first cannula 34, and the outflow conduit 18 is connected to the patient's vasculature, specifically, a second peripheral blood vessel 36 by way of a second cannula 38. The blood pump assembly 14 pumps blood from the first peripheral blood vessel 32, through the inflow conduit 16, through the blood pump assembly 14, and back into the second peripheral blood vessel 36 through the outflow conduit 18. In the illustrated embodiment, the first peripheral blood vessel 32 is a femoral vein, and the second peripheral blood vessel 36 is an axillary artery. It will be understood that the illustrated connections to the patient's vasculature are for illustrative purposes only, and that the blood pump assembly 14 may be connected to the patient's vasculature in any other suitable manner that enables that extracorporeal mechanical circulatory support system 10 to function as described herein, including, for example and without limitation, veno-venous (VV) connections and veno-arterial (VA) connections.

The controller 20 is communicatively coupled to the blood pump assembly 14, and is configured to control operation thereof. For example, the controller 20 is configured to control operation (e.g., a speed) of the blood pump 24. The controller 20 can generally include any suitable computer and/or other processing unit, including any suitable combination of computers, processing units and/or the like that may be communicatively coupled to one another (e.g., controller 20 can form all or part of a controller network). Thus, controller 20 can include one or more processor(s) and associated memory device(s) configured to perform a variety of computer-implemented functions (e.g., performing the methods, steps, calculations and/or the like disclosed herein). As used herein, the term “processor” refers not only to integrated circuits referred to in the art as being included in a computer, but also refers to a controller, a microcontroller, a microcomputer, a programmable logic controller (PLC), an application specific integrated circuit (ASIC), a digital signal processor (DSP), a field programmable gate array (FPGA), and other programmable circuits. Additionally, the memory device(s) of controller 20 may generally include memory element(s) including, but not limited to, non-transitory computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) can generally be configured to store suitable computer-readable instructions that, when implemented by the processor(s), configure the controller 20 to perform various functions including, but not limited to, controlling components of the blood pump assembly 14 as described herein.

The power source 22 provides power to the blood pump 24, controller 20, and other electrical components of the blood pump assembly 14, and may generally include any suitable power supply that enables the extracorporeal mechanical circulatory support system 10 to function as described herein. While the controller 20 and power supply are illustrated as being external to the blood pump assembly 14, all or part of the controller 20 and/or the power source 22 may be incorporated within the blood pump assembly 14 in other embodiments.

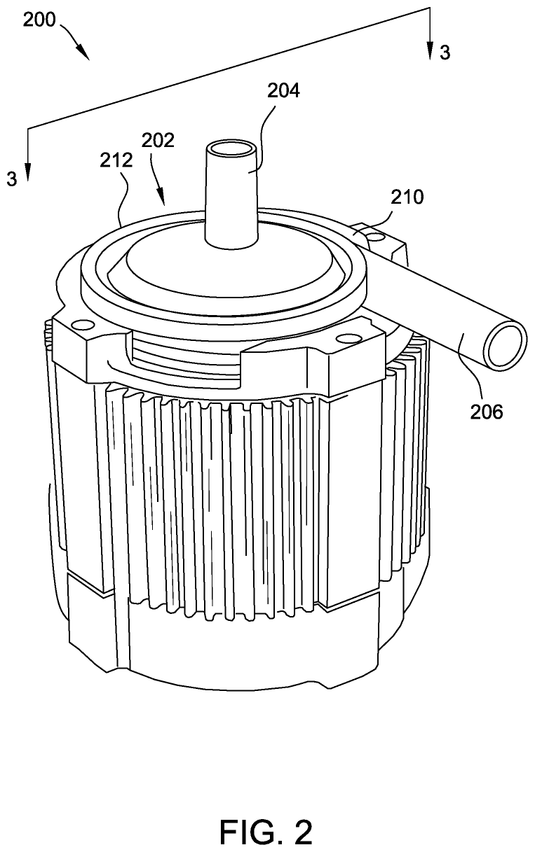

FIG. 2 is a perspective view of one embodiment of a blood pump assembly 200 that may be used to implement the blood pump assembly 14 (shown in FIG. 1). The blood pump assembly 200 includes a blood pump 202 having an inlet 204 for receiving blood from a patient's circulatory system, and an outlet 206 for delivering blood back to the patient's circulatory system. The inlet 204 and the outlet 206 may be fluidly connected to a patient's circulatory system using suitable fluid conduits (e.g., fluid conduits 16, 18, shown in FIG. 1), such as medical plastic tubing.

The blood pump assembly 200 is configured to pump blood from a patient's circulatory system such that blood is received at the inlet 204 and pumped out of the outlet 206 back into the patient's circulatory system. Along this flow path, the blood may be pumped through an oxygenator, such as the oxygenator 26 (shown in FIG. 1). The blood pump 202 and the oxygenator may be joined together in a non-permanent manner so as to allow the blood pump 202 and the oxygenator to be repeatedly joined and separated without damage. The blood pump 202 and the oxygenator may be removably connected to one another by any suitable non-permanent connecting means, including, for example and without limitation, threads, press-fit connectors, bayonet-type connectors, magnetic couplers, and combinations thereof. In other embodiments, the oxygenator may be non-removably connected to the blood pump 202, for example, by being integrally formed with the blood pump 202. The blood pump 202 includes a pump housing 210.

In the illustrated embodiment, the inlet 204 and the outlet 206 have a non-axial configuration, where the inlet 204 and the outlet 206 are not axially aligned with one another. More specifically, the blood pump 202 of this embodiment defines a volute 212 that redirects blood flowing in from the inlet 204 in a direction perpendicular to the direction of inflow. Alternatively, the inlet 204 and the outlet 206 may have an “in-line” configuration, where they are axially in-line with one another, or any other suitable configuration.

As blood flows from the patient 12, through the external blood pump assembly 200, and back to the patient, the blood is exposed to cooling (as the blood is outside of the patient 12). Generally, this cooling of the blood is undesirable. To counteract this cooling effect, in the systems and methods described herein, heat generated by the blood pump assembly 200 is used to heat the blood in a controlled manner. Specifically, operation of the blood pump assembly 200 is controlled to generate reactive power, which is used to gently heat the blood.

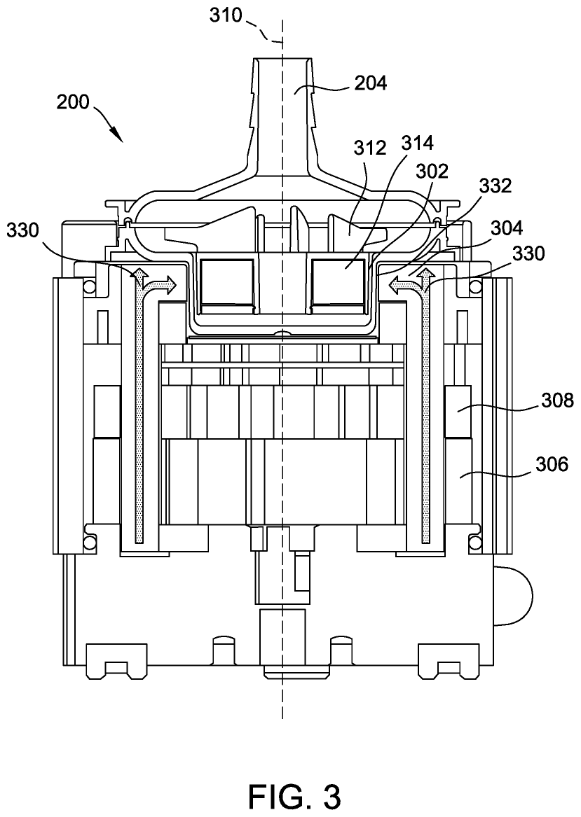

FIG. 3 is a cross-sectional view of the blood pump assembly 200 taken along the line 3-3 (shown in FIG. 2). As shown in FIG. 3, in this embodiment, the blood pump assembly 200 is a centrifugal pump that includes a rotor 302 and a stator 304. Further, the blood pump assembly 200 includes drive coils 306 and bearing coils 308. During operation, the rotor 302 is levitated magnetically with respect to the stator 304.

To operate the blood pump assembly 200, a controller (e.g., the controller 20 (shown in FIG. 1)) controls application of a drive current to the drive coils 306 and a bearing current to the bearing coils 308. The drive coils 306 and the bearing coils 308 may be, for example, copper coils. Running currents through the drive coils 306 and the bearing coils 308 causes a magnetic flux to be conducted by the stator 304, which is made of a magnetic material (e.g., a ferromagnetic material). The rotor 302 also includes one or more elements 314 made of a magnetic material (e.g., a permanent magnet).

Accordingly, the magnetic flux conducted through the stator 304 causes the rotor 302 to rotate, relative to the stator 304, about a longitudinal axis 310. A plurality of blades 312 are coupled to the rotor 302. As the rotor 302 rotates, the blades 312 rotate in turn, pumping blood through the blood pump assembly 200. As will be appreciated by those of skill in the art, the rotational speed of the rotor 302 and the position of the rotor 302 relative to the stator 304 is controlled by controlling the current flowing through the drive coils 306 and bearing coils 308.

Although the embodiments described herein are described in the context of a centrifugal pump with a magnetically levitated rotor, those of skill in the art will appreciate that the techniques disclosed herein may be implemented within any suitable blood pump assembly.

In the embodiments described herein, coils of the blood pump assembly 200 are powered using sinusoidal current and voltage waveforms. If the product of those two waveforms is zero or positive, only active power, P, is generated, resulting in an ideal power conversion. If, however, the product of the two waveforms is negative (which results when the waveforms are out of phase with one another), reactive power, Q, is generated.

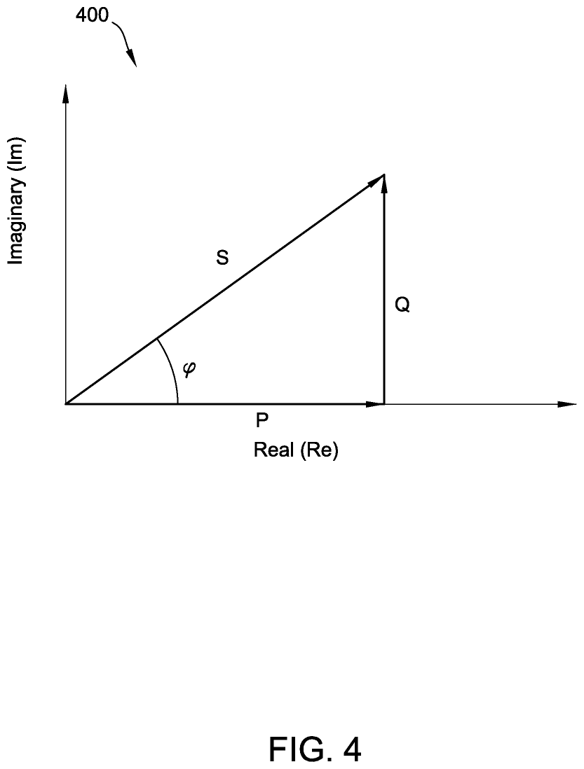

For example, FIG. 4 is a diagram 400 showing the relationship between the active power, P, the reactive power Q, and a total complex power, S. As shown by the diagram 400, the complex power is the vector sum of the active power and the reactive power. The apparent power is the magnitude of the complex power. Further, a phase angle, φ, between the voltage and current waveforms governs the values of the active power and the reactive power. For example, as shown by the diagram 400, if the phase angle is 0°, all power will be active power. In contrast, if the phase angle is 90°, all power will be reactive power.

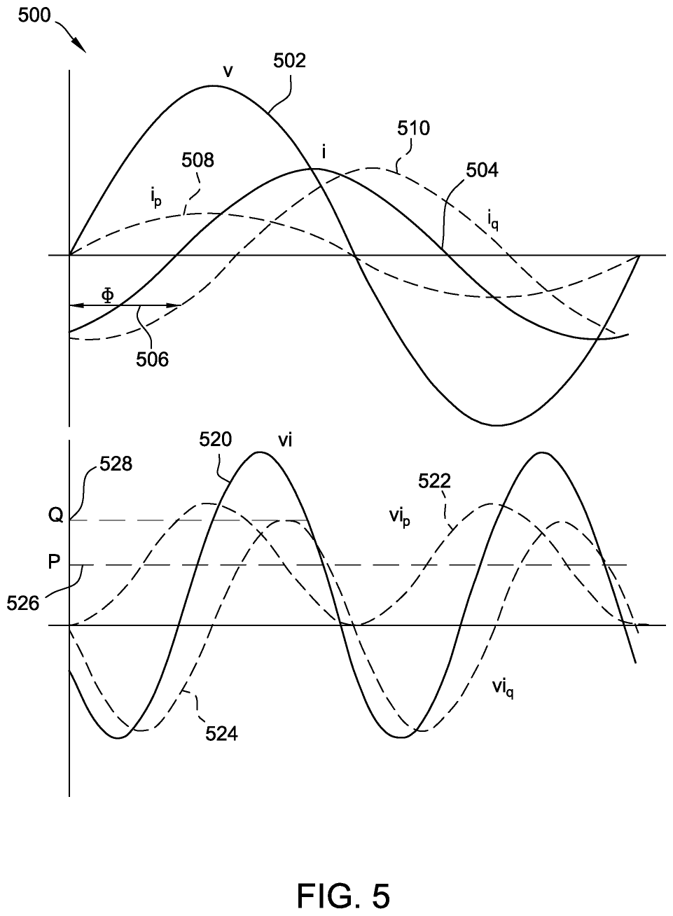

FIG. 5 is a diagram 500 illustrating the relationship between voltage, current, and the active and the reactive power. Specifically, the diagram 500 shows a sinusoidal voltage waveform 502 and a sinusoidal current waveform 504 separated by a phase angle 506, φ (corresponding to the zero-crossing shift between the voltage waveform 502 and the current waveform 504). The current waveform 504 has an active current component 508, iₚ, and a reactive current component 510, i_{q}.

The resulting power is the product of the voltage waveform 502 and the current waveform 504. For example, the diagram 500 shows an instantaneous power curve 520, vi, which has an active power component 522, viₚ, and a reactive power component 524, vi_{q}. This results in an active power value 526, P, and a reactive power value 528, Q. By controlling the phase angle 506, the active power value 526 and the reactive power value 528 can be adjusted.

Reactive power is generally undesirable, as the produced energy is usually converted into heat instead of being used to power the associated device. However, in the systems and methods described herein, reactive power is deliberately generated, as the resulting heat can be used to increase the temperature of the blood flowing through the system. That is, the current and voltage waveforms are purposely misaligned (e.g., using the controller) to generate reactive power for heating the blood.

Referring back to FIG. 3, the controller controls the phase angle between the voltage waveform and the current waveform to generate reactive power. In one embodiment, the controller adjusts the phase angle by controlling timing of switching circuitry that generates the voltage and current waveforms (e.g., controlling the timing at which one or more transistor switches are switched on or off). Alternatively, other suitable techniques may be used by the controller to adjust the phase angle.

The reactive power generated causes the drive coils 306 and/or the bearing coils 308 to generate heat. Further, the generated heat is directed along heat transfer paths 330 from the coils 306 and/or 308 towards blood transporting components (i.e., where blood is flowing). The heat is transferred into the blood through a heat transmission barrier 332. The heat transfer paths 330 may include a thermally conductive material to facilitate guiding the generated heat towards the heat transmission barrier 332.

When blood travels from the patient 12, through the blood pump assembly 200, and back to the patient 12, the blood may cool by approximately 2-3° Celsius. Accordingly, in one embodiment, the controller causes the blood pump assembly 200 to generate enough reactive power to heat the blood by approximately 2-3° Celsius, to counteract the cooling that would otherwise occur.

In some embodiments, reactive power generation (and consequently, heat) is controlled using a closed-loop approach. For example, one or more temperature sensors may be used to measure the blood temperature (e.g., at the blood pump assembly 200, and/or just before blood re-enters the patient 12). The controller receives the measured blood temperature, and automatically adjusts the phase angle between the voltage and current waveforms to increase or decrease the amount that the blood is heated as it passes through the blood pump assembly 200. Alternatively, the controller may adjust the phase angle based on a user input specifying a desired reactive power value and/or desired blood temperature increase value.

The systems and method described herein provide an extracorporeal blood circuit that includes a blood pump including an inlet, an outlet, and a rotor positioned between the inlet and the outlet, and a controller coupled to the blood pump. The controller is configured to drive rotation of the rotor using a voltage waveform and a current waveform, and adjust a phase angle between the voltage waveform and the current waveform to generate reactive power. The generated reactive power facilitates heating blood as the blood passes through the blood pump.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An extracorporeal blood circuit comprising:
a blood pump comprising an inlet, an outlet, and a rotor positioned between the inlet and the outlet; and
a controller coupled to the blood pump, the controller configured to:
drive rotation of the rotor using a voltage waveform and a current waveform; and
adjust a phase angle between the voltage waveform and the current waveform to generate reactive power, wherein the generated reactive power facilitates heating blood as the blood passes through the blood pump.

2. The extracorporeal blood circuit of claim 1, wherein the generated reactive power facilitates heating the blood by approximately 2-3° C. as the blood passes through the blood pump.

3. The extracorporeal blood circuit of claim 1, wherein the blood pump further comprises a stator and a plurality of coils, and wherein to drive rotation of the rotor, the controller is configured to drive current through the plurality of coils based on the voltage waveform and the current waveform, in order to cause the stator to conduct a magnetic flux.

4. The extracorporeal blood circuit of claim 3, wherein the generated reactive power heats the plurality of coils.

5. The extracorporeal blood circuit of claim 4, further comprising a thermally conductive material configured to channel heat from the plurality of coils to the blood.

6. The extracorporeal blood circuit of claim 1, further comprising at least one temperature sensor configured to measure a temperature of the blood.

7. The extracorporeal blood circuit of claim 6, wherein the controller is configured to adjust the phase angle based on the measured temperature of the blood.

8. The extracorporeal blood circuit of claim 1, wherein the controller is configured to adjust the phase angle based on a user input.

9. The extracorporeal blood circuit of claim 1, wherein the inlet of the blood pump is not axially aligned with the outlet of the blood pump.

10. A method of operating an extracorporeal blood circuit, the method comprising:
receiving, at an inlet of a blood pump, blood from a patient;
driving, using a controller, rotation of a rotor of the blood pump to pump the blood from the inlet of the blood pump to an outlet of the blood pump, wherein the controller drives rotation of the rotor using a voltage waveform and a current waveform; and
adjusting, using the controller, a phase angle between the voltage waveform and the current waveform to generate reactive power, wherein the generated reactive power facilitates heating the blood as the blood passes through the blood pump.

11. The method of claim 10, wherein the generated reactive power facilitates heating the blood by approximately 2-3° Celsius as the blood passes through the blood pump.

12. The method of claim 10, wherein the blood pump further includes a stator and a plurality of coils, and wherein driving rotation of the rotor comprises driving current through the plurality of coils based on the voltage waveform and the current waveform, in order to cause the stator to conduct a magnetic flux.

13. The method of claim 12, wherein the generated reactive power heats the plurality of coils.

14. The method of claim 13, further comprising channeling heat from the plurality of coils to the blood.

15. The method of claim 10, further comprising measuring, using at least one temperature sensor, a temperature of the blood.

16. The method of claim 15, wherein adjusting the phase angle comprises adjusting the phase angle based on the measured temperature of the blood.

17. The method of claim 10, wherein adjusting the phase angle comprises adjusting the phase angle based on a user input.

18. A controller for use in an extracorporeal blood circuit, the controller comprising:
a memory device; and
a processing device coupled to the memory device, the processing device configured to:
drive rotation of a blood pump based on a voltage waveform and a current waveform; and
adjust a phase angle between the voltage waveform and the current waveform to generate reactive power, wherein the generated reactive power facilitates heating blood as the blood passes through the blood pump.

19. The controller of claim 18, wherein the processor is configured to adjust the phase angle based on a measured temperature of the blood.

20. The controller of claim 18, wherein the processor is configured to adjust the phase angle based on a user input.
